(19) United States
(12) Patent Application Publication
(10) Pub. No.: US 2022/0261468 A1
(43) Pub. Date: Aug. 18, 2022

(54) EAR-BASED BIOMETRIC IDENTIFICATION

(71) Applicant: Starkey Laboratories, Inc., Eden Prairie, MN (US)

(72) Inventors: Andy S. Lin, Chanhassen, MN (US); Michael Karl Sacha, Chanhassen, MN (US); Peter Flanagan, St. Louis Park, MN (US)

(21) Appl. No.: 17/661,468

(22) Filed: Apr. 29, 2022

Related U.S. Application Data
(63) Continuation of application No. PCT/US2020/057977, filed on Oct. 29, 2020.
(60) Provisional application No. 62/929,491, filed on Nov. 1, 2019.

(51) Int. Cl. G06F 21/32; H04R 1/08; H04R 1/10; A61B 5/0295; A61B 5/00; G06F 21/62

(52) U.S. Cl. CPC G06F 21/32; H04R 1/08; H04R 1/1016; H04R 1/1041; H04R 2420/07; A61B 5/6817; A61B 5/6843; G06F 21/62; A61B 5/0295

(57) ABSTRACT

An ear-wearable device, such as a hearing aid or earbud, includes a plurality of biometric sensors configured to generate sensor measurements indicative of at least an aspect of contact between the housing of the ear-wearable device and the ear canal of the user, and compares the sensor measurements to a stored profile in order to determine whether an authorized user is wearing the ear-wearable device.

This application claims priority to U.S. Provisional Patent Application Ser. No. 62/929,491, entitled “EAR-BASED BIOMETRIC IDENTIFICATION,” and filed on Nov. 1, 2019, the entire content of which is incorporated herein by reference.

### TECHNICAL FIELD

This disclosure relates to hearing instruments.

### BACKGROUND

Hearing instruments are devices designed to be worn on, in, or near one or more of a user's ears. Common types of hearing instruments include hearing assistance devices (e.g., “hearing aids”), earbuds, headphones, hearables, cochlear implants, and so on. In some examples, a hearing instrument may be implanted or osseointegrated into a user. Some hearing instruments include additional features beyond just environmental sound-amplification. For example, some modern hearing instruments include advanced audio processing for improved device functionality, controlling and programming the devices, and beamforming, and some can even communicate wirelessly with external devices including other hearing instruments (e.g., for streaming media).

### SUMMARY

This disclosure describes techniques for identifying an authorized user or wearer of a hearing instrument, based on measurements indicative of one or more physical characteristics, such as a shape, of the user's ear canal. In some examples, an ear-wearable device includes a shell shaped for wearing in an ear of a user; a plurality of contact sensors coupled to the shell, each respective contact sensor of the plurality of contact sensors configured to generate first measurements indicative of an aspect of contact between the respective sensor and the ear of the user; and processing circuitry configured to determine whether an authorized user is wearing the ear-wearable device based at least in part on the first measurements.

In some examples, a method of determining whether an authorized user is wearing an ear-wearable device includes measuring, by a plurality of contact sensors disposed on a shell of the ear-wearable device, first measurements indicative of an aspect of contact between the respective contact sensor and an ear of a user; and determining whether the authorized user is wearing the ear-wearable device based at least in part on the first measurements.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a conceptual diagram illustrating an example system that includes one or more hearing instrument(s), in accordance with one or more techniques of this disclosure.

FIG. 2 is a block diagram illustrating example components of a hearing instrument, in accordance with one or more aspects of this disclosure.

FIG. 3 is a block diagram illustrating example components of a computing device, in accordance with one or more aspects of this disclosure.

FIG. 4A illustrates an example hearing instrument, in accordance with one or more aspects of this disclosure.

FIG. 4B illustrates the example hearing instrument of FIG. 4A within an ear of a user, in accordance with one or more aspects of this disclosure.

FIG. 5A is a cross-sectional view of an example hearing instrument inside the ear canal of a user, in accordance with one or more aspects of this disclosure.

FIG. 5B is a cross-sectional view of another example hearing instrument inside the ear canal of a user, in accordance with one or more aspects of this disclosure.

FIG. 6 is a flowchart illustrating an example operation in accordance with one or more example techniques described in this disclosure.

### DETAILED DESCRIPTION

Modern computing devices and systems include the capability to store and/or communicate sensitive (e.g., private) data. Accordingly, it is imperative that these devices and systems further include the capability to determine (e.g., verify) the identity of a user before the user is granted access to potentially sensitive data. A user's identity may be verified by information (e.g., a password) known only to the user, or by biometric identification. Biometric identification includes systems for identifying a user by measuring one or more unique physical characteristics of the user's body. For example, typical biometric authentication systems include fingerprint scanners, retinal scanners, and facial recognition systems. However, for many of these example verification systems, the user is required to actively (e.g., manually) enroll, such as by typing in a password or by swiping a fingerprint. In some examples in accordance with this disclosure, an ear-wearable device may include a plurality of sensors configured to automatically (e.g., passively) collect biometric data of a user's ear canal to identify and verify the user.

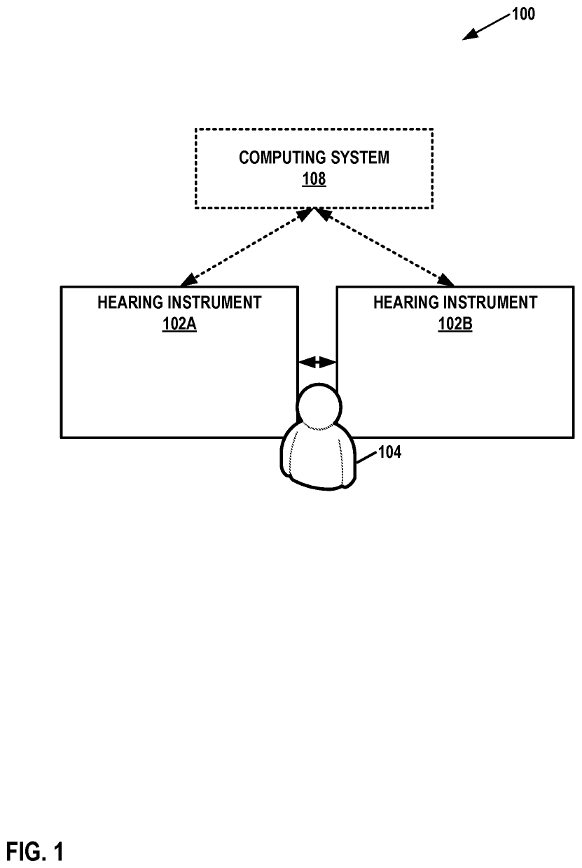

FIG. 1 is a conceptual diagram illustrating an example system 100 that includes hearing instruments 102A, 102B, in accordance with one or more techniques of this disclosure. This disclosure may refer to hearing instruments 102A and 102B collectively, as “hearing instruments 102.” A user 104 may wear hearing instruments 102. In some instances, such as when user 104 has unilateral hearing loss, user 104 may wear a single hearing instrument. In other instances, such as when user 104 has bilateral hearing loss, the user may wear two hearing instruments, with one hearing instrument for each ear of the user.

Hearing instruments 102 may comprise one or more of various types of devices that are configured to provide auditory stimuli to a user and that are designed for wear and/or implantation at, on, or near an ear of the user. Hearing instruments 102 may be worn, at least partially, in the ear canal or concha. One or more of hearing instruments 102 may include behind-the-ear (BTE) components that are worn behind the ears of user 104. In some examples, hearing instruments 102 comprise devices that are at least partially implanted into or osseointegrated with the skull of the user. In some examples, one or more of hearing instruments 102 is able to provide auditory stimuli to user 104 via a bone conduction pathway.

In any of the examples of this disclosure, each of hearing instruments 102 may comprise a hearing assistance device. Hearing assistance devices include devices that help a user hear sounds in the user's environment. Example types of hearing assistance devices may include hearing aid devices, Personal Sound-Amplification Products (PSAPs), cochlear implant systems (which may include cochlear implant magnets, cochlear implant transducers, and cochlear implant processors), and so on. In some examples, hearing instruments 102 are over-the-counter, direct-to-consumer, or prescription devices. Furthermore, in some examples, hearing instruments 102 include devices that provide auditory stimuli to the user that correspond to artificial sounds or sounds that are not naturally in the user's environment, such as recorded music, computer-generated sounds, or other types of sounds. For instance, hearing instruments 102 may include so-called “hearables,” earbuds, earphones, or other types of devices. Some types of hearing instruments provide auditory stimuli to the user corresponding to sounds from the user's environmental and also artificial sounds.

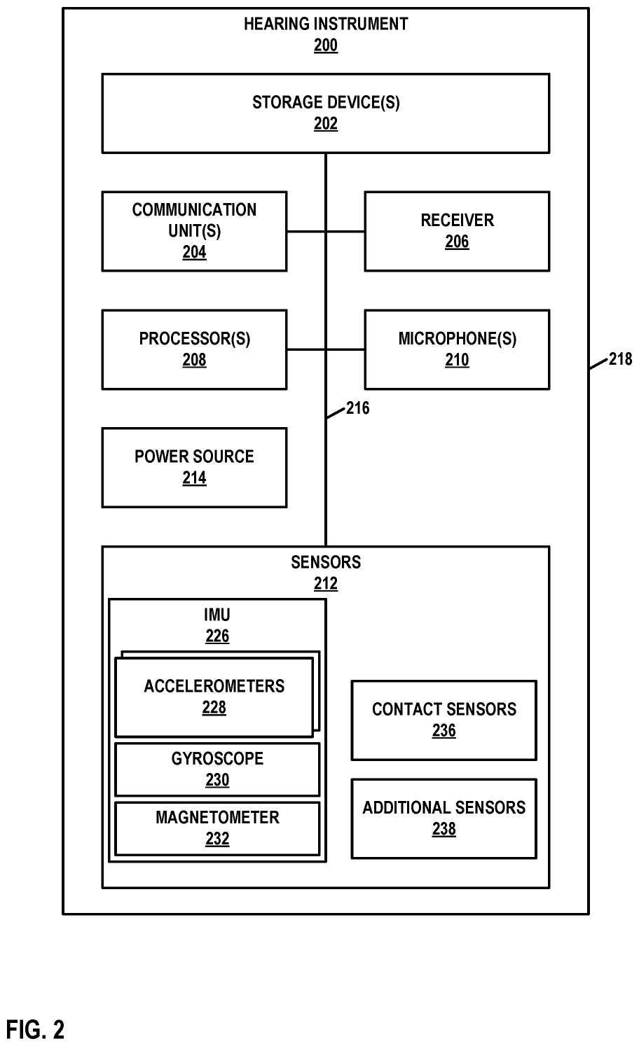

In some examples, one or more of hearing instruments 102 includes a housing or shell 218 (FIG. 2) that is designed to be worn in the ear for both aesthetic and functional reasons and encloses the electronic components of the hearing instrument. Such hearing instruments may be referred to as in-the-ear (ITE), in-the-canal (ITC), completely-in-the-canal (CIC), or invisible-in-the-canal (IIC) devices. In some examples, one or more of hearing instruments 102 may be behind-the-ear (BTE) devices, which include a housing worn behind the ear that contains all of the electronic components of the hearing instrument, including the receiver (i.e., the speaker). The receiver conducts sound to an earbud inside the ear via an audio tube. In some examples, one or more of hearing instruments 102 may be receiver-in-canal (RIC) hearing-assistance devices, which include a housing worn behind the ear that contains electronic components and a housing worn in the ear canal that contains the receiver.

Hearing instruments 102 may implement a variety of features that help user 104 hear better. For example, hearing instruments 102 may amplify the intensity of incoming sound, amplify the intensity of certain frequencies of the incoming sound, translate or compress frequencies of the incoming sound, and/or perform other functions to improve the hearing of user 104. In another example, hearing instruments 102 may implement a directional processing mode in which hearing instruments 102 selectively amplify sound originating from a particular direction (e.g., to the front of the user) while potentially fully or partially canceling sound originating from other directions. In other words, a directional processing mode may selectively attenuate off-axis unwanted sounds. The directional processing mode may help users understand conversations occurring in crowds or other noisy environments. In some examples, hearing instruments 102 may use beamforming or directional processing cues to implement or augment directional processing modes.

In some examples, hearing instruments 102 may reduce noise by canceling out or attenuating certain frequencies. Furthermore, in some examples, hearing instruments 102 may help user 104 enjoy audio media, such as music or sound components of visual media, by outputting sound based on audio data wirelessly transmitted to hearing instruments 102.

Hearing instruments 102 may be configured to communicate with each other. For instance, in any of the examples of this disclosure, hearing instruments 102 may communicate with each other using one or more wirelessly communication technologies. Example types of wireless communication technology include Near-Field Magnetic Induction (NFMI) technology, a 900 MHz technology, a BLUETOOTH™ technology, a WI-FI™ technology, audible sound signals, ultrasonic communication technology, infrared communication technology, an inductive communication technology, or another type of communication that does not rely on wires to transmit signals between devices. In some examples, hearing instruments 102 use a 2.4 GHz frequency band for wireless communication. In some examples, hearing instruments 102 may communicate with each other via non-wireless communication links, such as via one or more cables, direct electrical contacts, and so on.

As shown in the example of FIG. 1, system 100 may also include a computing system 108. In other examples, system 100 does not include computing system 108. Computing system 108 comprises one or more computing devices, each of which may include one or more processors. For instance, computing system 108 may comprise one or more mobile devices, server devices, personal computer devices, handheld devices, wireless access points, smart speaker devices, smart televisions, medical alarm devices, smart key fobs, smartwatches, smartphones, motion or presence sensor devices, smart displays, screen-enhanced smart speakers, wireless routers, wireless communication hubs, prosthetic devices, mobility devices, special-purpose devices, accessory devices, and/or other types of devices. Accessory devices may include devices that are configured specifically for use with hearing instruments 102. Example types of accessory devices may include charging cases for hearing instruments 102, storage cases for hearing instruments 102, media streamer devices, phone streamer devices, external microphone devices, remote controls for hearing instruments 102, and other types of devices specifically designed for use with hearing instruments 102. Actions described in this disclosure as being performed by computing system 108 may be performed by one or more of the computing devices of computing system 108. One or more of hearing instruments 102 may communicate with computing system 108 using wireless or non-wireless communication links. For instance, hearing instruments 102 may communicate with computing system 108 using any of the example types of communication technologies described elsewhere in this disclosure.

In some examples in accordance with this disclosure, hearing instruments 102 may include a plurality of contact sensors configured to collect biometric data measurements, in order to determine whether an authorized user is wearing the ear-wearable device (e.g., in order to verify the identity of user 104). In response to determining that user 104 is an authorized user of hearing instruments 102 and/or computing system 108, system 100 is configured to allow the authorized user access to data, such as by unlocking a device of computing system 108, enabling audio output through hearing instruments 102, or by enabling a digital currency transfer via computing system 108, as non-limiting examples.

FIG. 2 is a block diagram illustrating example components of hearing instrument 200, in accordance with one or more aspects of this disclosure. Hearing instrument 200 may be either one of hearing instruments 102. In the example of FIG. 2, hearing instrument 200 comprises one or more storage devices 202, one or more communication unit(s) 204, a receiver 206, one or more processor(s) 208, one or more microphone(s) 210, a set of sensors 212, a power source 214, and one or more communication channels 216. Communication channels 216 provide communication between storage devices 202, communication unit(s) 204, receiver 206, processor(s) 208, a microphone(s) 210, and sensors 212. Components 202, 204, 206, 208, 210, and 212 may draw electrical power from power source 214.

In the example of FIG. 2, each of components 202, 204, 206, 208, 210, 212, 214, and 216 are contained within a single housing 218. However, in other examples of this disclosure, components 202, 204, 206, 208, 210, 212, 214, and 216 may be distributed among two or more housings. For instance, in an example where hearing instrument 200 is a RIC device, receiver 206 and one or more of sensors 212 may be include in an in-ear housing separate from a behind-the-ear housing that contains the remaining components of hearing instrument 200. In such examples, a RIC cable may connect the two housings.

Furthermore, in the example of FIG. 2, sensors 212 include an inertial measurement unit (IMU) 226 that is configured to generate data regarding the motion of hearing instrument 200. IMU 226 may include a set of sensors. For instance, in the example of FIG. 2, IMU 226 includes one or more of accelerometers 228, a gyroscope 230, a magnetometer 232, combinations thereof, and/or other sensors for determining the motion of hearing instrument 200. Furthermore, in the example of FIG. 2, hearing instrument 200 may include one or more contact sensors 236 and additional sensors 238. Contact sensors 236 may include electrodes and/or piezoelectric sensors, for example. Additional sensors 236 may include photoplethysmography (PPG) sensors, blood oximetry sensors, blood pressure sensors, electrocardiograph (EKG) sensors, body temperature sensors, electroencephalography (EEG) sensors, environmental temperature sensors, environmental pressure sensors, environmental humidity sensors, skin galvanic response sensors, microphones, and/or other types of sensors.

In some examples in accordance with this disclosure, system 100 (FIG. 1), including at least one hearing instrument 200, is configured to determine whether an authorized user is wearing hearing instrument 200 (e.g., identify user 104) (FIG. 1) based at least in part on biometric data measurements collected by one or more contact sensors 236. For example, contact sensors 236 may include a plurality of electrodes coupled to (e.g., positioned on or embedded within) housing 218 of hearing instrument 200. The electrodes may be configured to collect biometric data measurements indicative of a relative amount of contact between housing 218 and the skin of the ear canal of user 104. For example, the biometric measurements may indicate a magnitude of at least one aspect of contact (e.g., a magnitude of force, pressure, surface area, etc.) detected by each electrode and/or the ratios of the amounts of contact detected by any two or more electrodes.

System 100 may then determine whether an authorized user is wearing hearing instrument 200, based at least in part on the contact sensor measurements. For example, system 100 may compare the contact sensor measurements to a stored profile associated with an authorized user. For example, a stored profile may include a set of previous biometric measurements indicative of the shape of the ear canal of an authorized user. System 100 may compare the current biometric measurements to the stored profile to determine whether the current biometric measurements “match” (e.g., are within a threshold tolerance from) the previous biometric measurements of the stored profile.

If the current measurements match the stored profile, system 100 determines that the current user 104 is an authorized user of hearing instrument 200 (e.g., identifies user 104). If the current measurements do not match the stored profile, system 100 determines that the current user 104 is not an authorized user of hearing instrument 200 (e.g., fails to identify user 104).

In another example, hearing instrument 200 may be in data communication with a neural network trained to determine whether the current user of hearing instrument 200 is an authorized user, based at least in part on data input that includes the contact sensor measurements. In other examples, hearing instrument 200 and sensors 212 may include more, fewer, or different components.

Storage devices 202 may store data. Storage devices 202 may comprise volatile memory and may therefore not retain stored contents if powered off. Examples of volatile memories may include random access memories (RAM), dynamic random access memories (DRAM), static random access memories (SRAM), and other forms of volatile memories known in the art. Storage devices 202 may further be configured for long-term storage of information as non-volatile memory space and retain information after power on/off cycles. Examples of non-volatile memory configurations may include magnetic hard discs, optical discs, flash memories, or forms of electrically programmable memories (EPROM) or electrically erasable and programmable (EEPROM) memories. In some examples, storage devices 202 are configured to store data, such as a profile indicative of a shape of the ear canal of an authorized user, such that processor(s) 208 may compare biometric measurements collected by contact sensors 236 to the stored profile in order to determine whether user 104 is an authorized user of hearing instrument 200.

Communication unit(s) 204 may enable hearing instrument 200 to send data to and receive data from one or more other devices, such as a device of computing system 108 (FIG. 1), another hearing instrument, an accessory device, a mobile device, or another type of device. Communication unit(s) 204 may enable hearing instrument 200 using wireless or non-wireless communication technologies. For instance, communication unit(s) 204 enable hearing instrument 200 to communicate using one or more of various types of wireless technology, such as a BLUETOOTH™ technology, 3G, 4G, 4G LTE, 5G, ZigBee, Wi-Fi™, Near-Field Magnetic Induction (NFMI), ultrasonic communication, infrared (IR) communication, or another wireless communication technology. In some examples, communication unit(s) 204 may enable hearing instrument 200 to communicate using a cable-based technology, such as a Universal Serial Bus (USB) technology.

Receiver 206 comprises one or more speakers for generating audible sound. Microphone(s) 210 detects incoming sound and generates one or more electrical signals (e.g., an analog or digital electrical signal) representing the incoming sound.

Processor(s) 208 may be processing circuits configured to perform various activities. For example, processor(s) 208 may process the signal generated by microphone(s) 210 to enhance, amplify, or cancel-out particular channels within the incoming sound. Processor(s) 208 may then cause receiver 206 to generate sound based on the processed signal. In some examples, processor(s) 208 include one or more digital signal processors (DSPs). In some examples, processor(s) 208 may cause communication unit(s) 204 to transmit one or more of various types of data. For example, processor(s) 208 may cause communication unit(s) 204 to transmit data to computing system 108. Furthermore, communication unit(s) 204 may receive audio data from computing system 108 and processor(s) 208 may cause receiver 206 to output sound based on the audio data.

In some examples in accordance with this disclosure, processor(s) 208 may be configured to compare biometric measurements collected by contact sensors 236 to a profile stored within memory 202 and determine, based on the comparison, whether user 104 is an authorized user of hearing instrument 200 (e.g., to identify user 104). In some examples, processor(s) 208 may be configured to perform one or more actions in response to determining that user 104 is an authorized user (e.g., in response to identifying user 104). For example, processor(s) 208 may be configured to allow user 104 access to data via one or more devices, in response to determining that user 104 is an authorized user. For example, processor(s) 208 may be configured to activate the functionality of hearing instrument 200 (e.g., enable receiver 206 to output audio data to user 104) in response to identifying user 104. In other examples, processor(s) 208 may be configured to enable a data transfer between two or more devices, such as between hearing instrument 200 and computing device 108, in response to identifying user 104. In other examples, processor(s) 208 may be configured to enable a data transfer between two computing devices, such as between a computing device of computing system 108 and a second computing device, in response to identifying user 104. For example, processor(s) 208 may be configured to authorize a digital payment via computing device 300 in response to identifying user 104. In other examples, processor(s) 208 may be configured to unlock (e.g., enable access to) computing device 108, such as a smartphone, laptop, tablet, or other computing device, in response to identifying user 104.

Conversely, processor(s) 208 may be configured to compare biometric measurements collected by contact sensors 236 to a profile stored within memory 202, and determine, based on the comparison that current user 104 is not an authorized user of hearing instrument 200 (e.g., to fail to identify user 104). In some examples, processor(s) 208 may be configured to perform an action, such as preventing user 104 from accessing data via one or more devices, in response to failing to identify user 104. For example, processor(s) 208 may be configured to deactivate the functionality of hearing instrument 200 (e.g., disable receiver 206 from outputting audio data to user 104) in response to failing to identify user 104. In other examples, processor(s) 208 may be configured to disable a data transfer between two or more devices, such as between hearing instrument 200 and computing device 108, in response to failing to identify user 104. In other examples, processor(s) 208 may be configured to disable a data transfer between two computing devices, such as between a computing device of computing system 108 and a second computing device, in response to failing to identify user 104. For example, processor(s) 208 may be configured to block, disable, de-authorize, or otherwise or prevent a digital payment via computing device 300 in response to failing to identify user 104. In other examples, processor(s) 208 may be configured to lock (e.g., revoke access to) computing device 108, in response to failing to identify user 104.

In this way, the techniques of this disclosure provide technical solutions to one or more technical problems. For example, by preventing hearing instrument 102A from functioning for an unauthorized user, the techniques of this disclosure may deter theft of expensive hearing aids and/or earbuds, which may typically cost up to hundreds or thousands of dollars. In a similar manner, the techniques of this disclosure provide an additional layer of security for computing device 300. Additionally, unlike typical authentication methods, which require a user to actively swipe a fingerprint or type in a passcode, the techniques of this disclosure may provide a continuous, automatic, or “smooth” biometric authentication method that does not require a user to pause what they are doing in order to verify their identity as long as they are wearing hearing instruments 102.

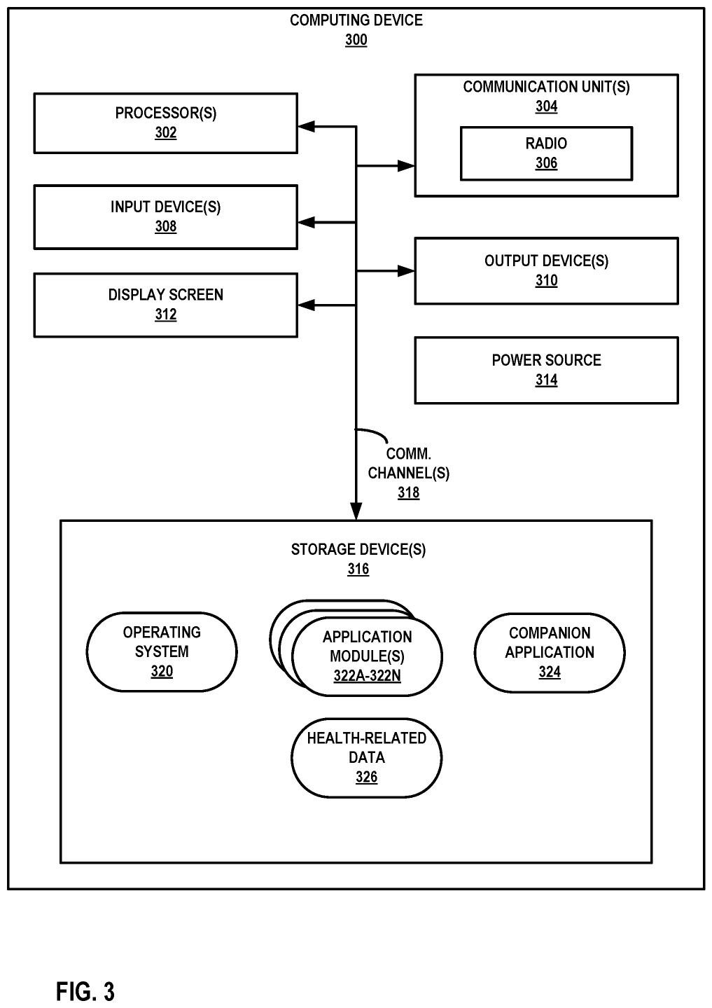

FIG. 3 is a block diagram illustrating example components of computing device 300, in accordance with one or more aspects of this disclosure. FIG. 3 illustrates only one particular example of computing device 300, and many other example configurations of computing device 300 exist. Computing device 300 may be a computing device in computing system 108 (FIG. 1).

As shown in the example of FIG. 3, computing device 300 includes one or more processor(s) 302, one or more communication unit(s) 304, one or more input device(s) 308, one or more output device(s) 310, a display screen 312, a power source 314, one or more storage device(s) 316, and one or more communication channels 318. Computing device 300 may include other components. For example, computing device 300 may include physical buttons, microphones, speakers, communication ports, and so on. Communication channel(s) 318 may interconnect each of components 302, 304, 308, 310, 312, and 316 for inter-component communications (physically, communicatively, and/or operatively). In some examples, communication channel(s) 318 may include a system bus, a network connection, an inter-process communication data structure, or any other method for communicating data. Power source 314 may provide electrical energy to components 302, 304, 308, 310, 312 and 316.

Storage device(s) 316 may store information required for use during operation of computing device 300. In some examples, storage device(s) 316 have the primary purpose of being a short term and not a long-term computer-readable storage medium. Storage device(s) 316 may be volatile memory and may therefore not retain stored contents if powered off. Storage device(s) 316 may further be configured for long-term storage of information as non-volatile memory space and retain information after power on/off cycles. In some examples, processor(s) 302 on computing device 300 read and may execute instructions stored by storage device(s) 316.

Computing device 300 may include one or more input device(s) 308 that computing device 300 uses to receive user input. Examples of user input include tactile, audio, and video user input. Input device(s) 308 may include presence-sensitive screens, touch-sensitive screens, mice, keyboards, voice responsive systems, microphones or other types of devices for detecting input from a human or machine.

Communication unit(s) 304 may enable computing device 300 to send data to and receive data from one or more other computing devices (e.g., via a communications network, such as a local area network or the Internet). For instance, communication unit(s) 304 may be configured to receive source data exported by hearing instrument(s) 102, receive comment data generated by user 112 of hearing instrument(s) 102, receive and send request data, receive and send messages, and so on. In some examples, communication unit(s) 304 may include wireless transmitters and receivers that enable computing device 300 to communicate wirelessly with the other computing devices. For instance, in the example of FIG. 3, communication unit(s) 304 include a radio 506 that enables computing device 300 to communicate wirelessly with other computing devices, such as hearing instruments 102 (FIG. 1). Examples of communication unit(s) 304 may include network interface cards, Ethernet cards, optical transceivers, radio frequency transceivers, or other types of devices that are able to send and receive information. Other examples of such communication units may include BLUETOOTH™, 3G, 4G, 5G, and WI-FI™ radios, Universal Serial Bus (USB) interfaces, etc. Computing device 300 may use communication unit(s) 304 to communicate with one or more hearing instruments (e.g., hearing instrument 102 (FIG. 1, FIG. 2)). Additionally, computing device 300 may use communication unit(s) 304 to communicate with one or more other remote devices.

Output device(s) 310 may generate output. Examples of output include tactile, audio, and video output. Output device(s) 310 may include presence-sensitive screens, sound cards, video graphics adapter cards, speakers, liquid crystal displays (LCD), or other types of devices for generating output.

Processor(s) 302 may read instructions from storage device(s) 316 and may execute instructions stored by storage device(s) 316. Execution of the instructions by processor(s) 302 may configure or cause computing device 300 to provide at least some of the functionality ascribed in this disclosure to computing device 300. As shown in the example of FIG. 3, storage device(s) 316 include computer-readable instructions associated with operating system 320, application modules 322A-322N (collectively, “application modules 322”), and a companion application 324. Additionally, in the example of FIG. 3, storage device(s) 316 may store health-related data 326.

Execution of instructions associated with operating system 320 may cause computing device 300 to perform various functions to manage hardware resources of computing device 300 and to provide various common services for other computer programs. Execution of instructions associated with application modules 322 may cause computing device 300 to provide one or more of various applications (e.g., “apps,” operating system applications, etc.). Application modules 322 may provide particular applications, such as text messaging (e.g., SMS) applications, instant messaging applications, email applications, social media applications, text composition applications, and so on.

Execution of instructions associated with companion application 324 by processor(s) 302 may cause computing device 300 to perform one or more of various functions. For example, execution of instructions associated with companion application 324 may cause computing device 300 to configure communication unit(s) 304 to receive data from hearing instruments 102 and use the received data to present health-related data to a user, such as user 104 or a third-party user. In some examples, companion application 324 is an instance of a web application or server application. In some examples, such as examples where computing device 300 is a mobile device or other type of computing device, companion application 324 may be a native application.

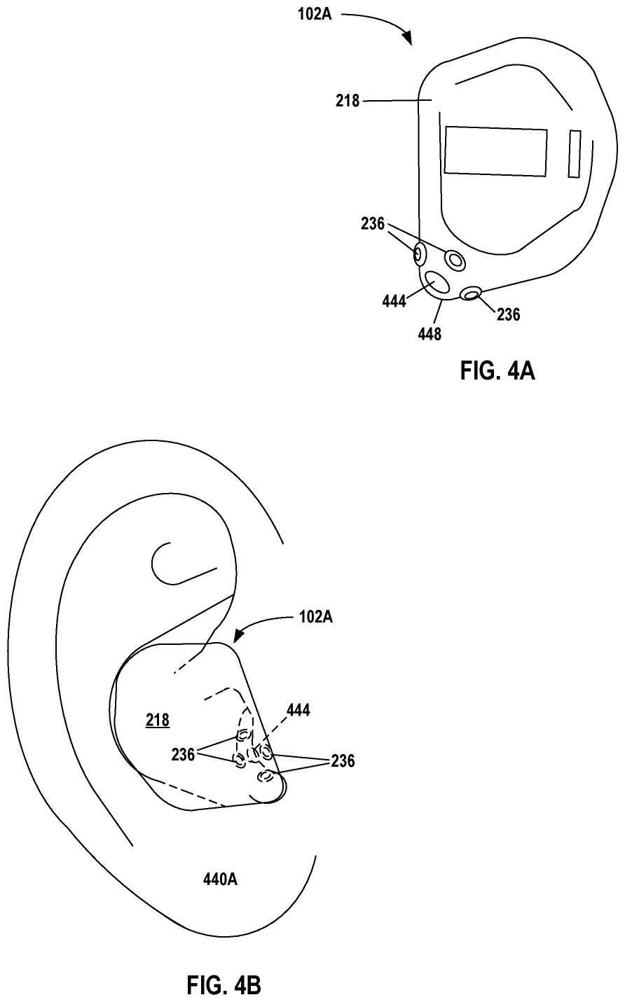

FIG. 4A illustrates an example hearing instrument 102A, in accordance with one or more aspects of this disclosure. Specifically, FIG. 4A depicts a medial side (e.g., a side configured to contact a right ear 440A (FIG. 4B) of a user 104 (FIG. 1)) of hearing instrument 102A. As depicted in FIG. 4A, hearing instrument 102A includes a housing or shell 218, a protrusion 448, and one or more contact sensors 236 and/or additional sensors 238. Although FIG. 4A depicts hearing instrument 102A, hearing instrument 102A may include any ear-wearable device, such as an earbud, headphones, etc.

Shell 218 defines a protrusion 448. Protrusion 448 is configured to fit within an ear canal of a user 104 (FIG. 1). Protrusion 448 defines an opening 444 through which one or more speakers of receiver 206, housed within shell 218, transmit sound into the ear canal of user 104. In some examples in accordance with this disclosure, protrusion 448 includes a plurality of contact sensors 236, either positioned on top of shell 218 or embedded within (e.g., level or flush with) shell 218. Although FIG. 4A depicts three contact sensors 236, protrusion 448 may include any number of contact sensors and/or additional sensors 238 (FIG. 2). In some examples, one or more of contact sensors 236 may be located elsewhere on shell 218 (e.g., other than on protrusion 448). For example, sensors 236 and/or 238 may be located on a “seal area” of shell 218, wherein shell 218 contacts the ear canal to form an acoustic seal.

Contact sensors 236 and additional sensors 238 may include any devices configured to generate biometric measurements indicative of at least one physical characteristic of the ear canal of user 104 (e.g., size and/or shape). For example, contact sensors 236 may include a plurality of electrodes configured to measure at least one aspect of contact (e.g., an electrical impedance that is proportional to an amount of force, pressure, or surface area) between shell 218 and the skin of the ear canal of user 104 when hearing instrument 102A is worn within the ear 440A (FIG. 4B) of user 104. Similarly, contact sensors 236 may include a plurality of piezoelectric sensors configured to measure at least one aspect of contact (e.g., a voltage or current that is proportional to an amount of force, pressure, and/or surface area) between shell 218 and the ear canal of user 104.

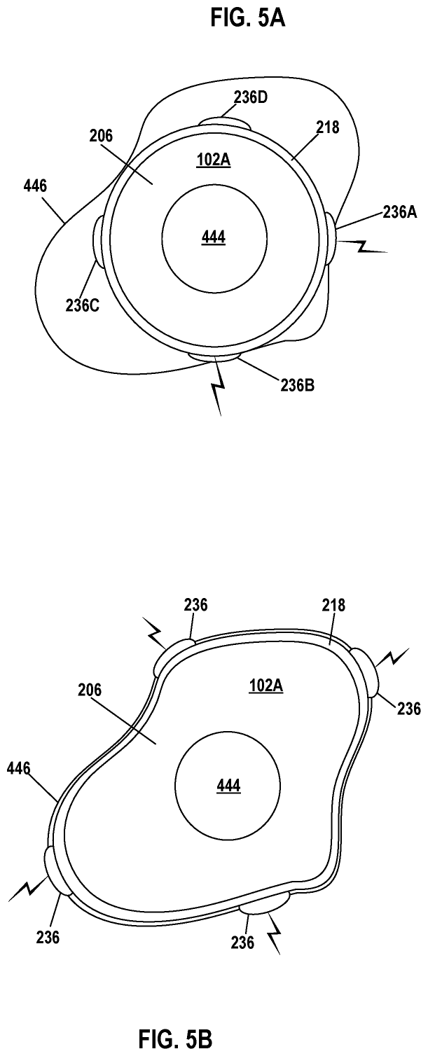

For example, depending on the difference between the unique size and/or shape of the ear canal of user 104 and the shape of shell 218 along protrusion 448, each contact sensor 236 may measure a different relative amount of contact between shell 218 (e.g., between the respective contact sensor 236, which may be approximately aligned with shell 218) and the user's ear canal. As depicted in greater detail in FIG. 5A, for a shell 218 having a substantially different shape (circular) than the unique shape of the user's ear canal 446 (oblong), some contact sensors (236A, 236B) may measure a relatively greater amount of contact, while other contact sensors (236C, 236D) may measure relatively less contact or even no contact at all.

During an initial set-up process, an authorized user of hearing instruments 102 may create a profile, including a dataset of measurements collected from contact sensors 236, describing the set of relative amounts of contact between each of sensors 236 and ear canal 446 that is unique to the authorized user. Hearing instruments 102 may store the profile in memory 202 (FIG. 2).

FIG. 4B illustrates the example hearing instrument 102A of FIG. 4A worn within an ear of a user, in accordance with one or more aspects of this disclosure. When hearing instrument 102A is worn within an ear 440A of a user 104, as shown in FIG. 4B, hearing instrument 102A (e.g., processor(s) 208 within hearing instrument 102A) is configured to collect (e.g., receive) current contact sensor measurements from sensors 236, and compare the current sensor measurements to the authorized user's profile stored within memory 202.

For example, hearing instrument 102A may be configured to collect and compare contact sensor measurements essentially continuously while instrument 102A is worn within an ear 440A (for example, as long as any one contact sensor 236 detects any amount of contact with ear canal 446). In other examples, hearing instrument 102A may be configured to periodically collect and compare sensor measurements, such as every few seconds or minutes. In other examples, hearing instrument 102A may collect and compare sensor measurements based on an intended data transfer between two devices. For example, hearing instrument 102A may collect and compare sensor measurements before any audio data is transferred to, and/or output from, speakers of receiver 206. In other examples, hearing instrument 102A may collect and compare sensor measurements in response to an action (such as a button pressed or touchscreen activated), associated with computing device 300 (FIG. 3).

If processor(s) 208 determine that the current contact sensor measurements and the authorized user's stored profile match (e.g., respective contact sensor measurements are within a determined threshold of one another), then processor(s) 208 identify user 104 as an authorized user of hearing instrument 102A, and may accordingly perform an action to allow user 104 access to data via one or more devices. For example, processor(s) 208 may activate the audio-output capabilities of receiver 206, if the audio-output capabilities of receiver 206 are not activated already. In another example, processor(s) 208 may enable a data transfer between hearing instrument 102A and an enrolled (e.g., authorized or recognized) computing device 300. In other examples, processor(s) 208 may be configured to activate or “unlock” computing device 300. For example, computing device 300 may include a smartphone or other personal computer that may remain unlocked as long as processor(s) 208 recognize the biometric sensor data. A device may be said to be “unlocked” when no further user authentication is necessary in order to access specific functionality of the device, such as an application, program, or other functionality.

In some examples, the unique shape of the user's ear canal 446 may not be substantially static, and may change in response to any number of factors including time (e.g., growth with aging), temperature, movement of the user's jaw or eyes, swelling (e.g., in response to injury or illness), or other factors. Any or all of these factors may cause current sensor measurements to misalign with the stored profile, even for an authorized user, at any given time. However, in some examples, the shape of the user's ear canal 446, while dynamic, may change in a predictable or consistent way that may nevertheless be useful for biometric identification. For example, hearing instrument 102A may prompt user 104 to perform a specific, pre-determined movement of the user's jaw, such as by speaking the user's name or other verbal phrase. Each of sensors 236 may not only be configured to measure an instantaneous aspect of contact, but also a change in an aspect of contact over time while user 104 performs the specific jaw movement. In these examples, processor(s) 208 may be configured to determine whether an authorized user is wearing hearing instrument 102A based at least in part on both the measurements of the instantaneous aspect of contact and the change in the aspect of contact over time. For example, processor(s) 208 may compare the collected sensor measurements with a stored profile indicative of a change in an aspect of contact over time for an authorized user.

In some examples, additionally or alternatively to contact sensors 236 (e.g., electrodes or piezoelectric sensors), one or more additional sensors 238 may be configured to measure other unique biometric characteristics of user 104, such as to determine whether hearing instrument 102 is correctly placed within the ear of user 104 (e.g., to provide more accurate data) and/or to provide additional data with which to identify user 104. For example, a greater number and/or types of sensor data that collected about user 104 significantly reduces that possibility that an unauthorized user will have similar-enough biometric measurements (e.g., within the pre-determined threshold tolerances) that will match the authorized user's profile.

For example, a speaker of receiver 206 may be configured to output an emitted tone or sound, such as a series of narrow-band, short-duration chirps, into the user's ear canal. The emitted sound may include audible sound (e.g., between approximately 20 Hz-20 kHz), or may be outside the human-audible frequency range (e.g., below 20 Hz or above 20 kHz). In these examples, one or more of additional sensors 238 may include a microphone configured to detect a reflection of the emitted sound (e.g., a reflected sound), which may include at least one measurable audio characteristic that differs from the original emitted sound based on the unique physical characteristics (e.g., size and/or shape) of the user's ear canal. For example, processor(s) 208 may be configured to determine, based on the reflected sound detected by the microphone, one or more audio characteristics such as a unique detection-time delay between the emitted sound and the reflected sound, a frequency spread of the reflected sound, a resonance of the reflected sound, or an amplitude attenuation between the emitted sound and the reflected sound. Processor(s) 208 may compare the audio characteristic to a corresponding characteristic stored in the authorized user's profile. If the currently detected audio characteristic matches the characteristic stored in the authorized user's profile (e.g., respective audio characteristics are within a threshold tolerance of one another), then system 100 (e.g., hearing devices 102 and/or computing system 108 of FIG. 1) identifies user 104 as an authorized user, and in response, may enable user-access to data via one or more devices, as described above.

In some examples, one or more of additional sensors 238 may be configured to measure a capacitive coupling with the skin of ear canal 446, for example, based at least in part on the unique distance between each respective sensor 238 and the skin of ear canal 446. In the same manner as the contact sensor measurements, the capacitive coupling measurements may be stored within the authorized user's profile and may be used to determine whether the current user 104 is an authorized user.

In some examples, one or more of additional sensors 238 may include a photoplethysmography (PPG) sensor configured to measure a blood perfusion and/or heartbeat under the skin at a respective location in the user's ear. In some examples, processor(s) 208 may determine whether hearing instrument 102 is correctly placed within the user's ear based at least in part on the PPG sensor measurements. For example, PPG sensor(s) may each detect a standard-range blood perfusion when hearing instrument 102 is correctly worn within the ear.

In some examples, PPG sensors may also measure a blood perfusion for a user. System 100 may use PPG sensor measurements to determine whether hearing instrument 102 is incorrectly placed or misaligned within an ear of user 104. For example, PPG sensor measurements indicate an above-threshold signal-to-noise ratio, processor(s) 208 may be configured to output an indication to user 104 that the user cannot be identified due to hearing-instrument misalignment.

In another example, PPG sensor 238 may be configured to measure a heartbeat of a user. When hearing instrument 102 is correctly positioned within the user's ear, PPG sensor 238 may detect a heartbeat having a relatively low signal-to-noise ratio, for example, below a determined threshold signal-to-noise ratio. However, if PPG sensor 238 detects an above-threshold signal-to-noise ratio, hearing instrument 102 may be configured to output an indication to user 104 that they cannot be identified due to incorrect placement of hearing instrument 102 within the ear. These examples may help to conserve battery power for hearing instrument 102, which would otherwise waste energy collecting misaligned sensor measurements. These examples may also provide an added layer of security by ensuring a correct fit within an authorized user's ear before collecting contact sensor measurements.

In some examples, two or more of additional sensors 238 may include temperature sensors configured to measure a respective temperature at a location on or in the user's ear, such that processor(s) 208 may determine a temperature gradient between the two or more temperature sensors. In some examples, processor(s) 208 may determine whether hearing instrument 102 is correctly placed within the user's ear based at least in part on the temperature gradient. For example, a temperature sensor disposed on protrusion 448 may indicate a temperature near human-body temperature when hearing instrument 102 is worn within the ear, whereas a temperature sensor disposed outside of the ear canal may indicate a temperature nearer the ambient temperature..

In examples in which hearing instrument 102 includes multiple (e.g., two or more) different types of additional sensors 238, the different types of sensors 238 may be used in combination as extra layers of identification security. A hearing instrument having a combination of different types of sensors may substantially complicate an attempt to “fake” a user authentication, for example, by ensuring that the hearing instrument is actually being worn within an ear of a user, such that it may be more difficult to artificially simulate the contact sensor measurements. In some examples, system 100 may be configured to require that every different type of additional sensor data (e.g., data collected by different types of additional sensors 238) individually identifies user 104 in order for system 100 to permit user access to data, as described above. In other examples, system 100 may be configured to require that at least one type of additional sensor data must positively identify user 104 in order for system 100 to permit user access. In other examples, system 100 may be configured to require that a majority of the different types of additional sensor data individually identifies user 104 before system 100 may permit user access to data.

In some examples, one or more of contact sensors 236 and/or additional sensors 238 may include a surface texture configured to push away ear wax or other debris in order to improve contact between sensors 236, 238 and the skin of ear canal 446.

FIGS. 5A and 5B are cross-sectional views depicting two different example hearing instruments inside the ear canal 446 of a user, in accordance with one or more aspects of this disclosure. Specifically, FIG. 5A depicts a cross-section of protrusion 448 having a “standardized” shape (e.g., a common cross-sectional area irrespective of the particular user 104), whereas FIG. 5B depicts a cross-section of a protrusion 448 having a “customized” shape (e.g., a cross-sectional area customized to closely fit the shape of ear canal 446 for one particular user 104). While the two examples are functionally equivalent, the only noticeable distinction may be found in the nature of the stored profiles. For the “standardized” example depicted in FIG. 5A, the stored profile may indicate measurements of relative contact between each of sensors 236. As described above, for example, sensors 236A and 236B may measure a positive amount of contact, whereas sensors 236C and 236D may measure little or no contact. Conversely, because “customized” shell 218 of FIG. 5B is designed to closely match the shape of ear canal 446, each of contact sensors 236 is more likely to measure a relatively uniform amount of contact with ear canal 446. For example, contact sensors 236 on shell 218 will measure different relative amounts of contact for a user 104 for whom a shell 218 is customized, as compared to a different user 104 for whom shell 218 is not customized. Accordingly, system 100 may use this distinction between different relative amounts of contact for different users to identify a single authorized user for whom hearing instrument 102 is customized.

In some other examples not depicted in FIGS. 5A or 5B, shell 218 may also be “semi-customized,” in which user 104 selects, from among a limited number of different shell shapes, the one that is most comfortable for the particular user. In these examples, although multiple users may use similar-shaped hearing instruments 102, because the size and shape of each user's ear canal is unique, the relative amounts of contact between the respective users' ear canals and the shells 218 will similarly be unique. Accordingly, system 100 may exploit this unique profile (e.g., authorized user's profile comprising relative contact measurements) to identify an authorized user.

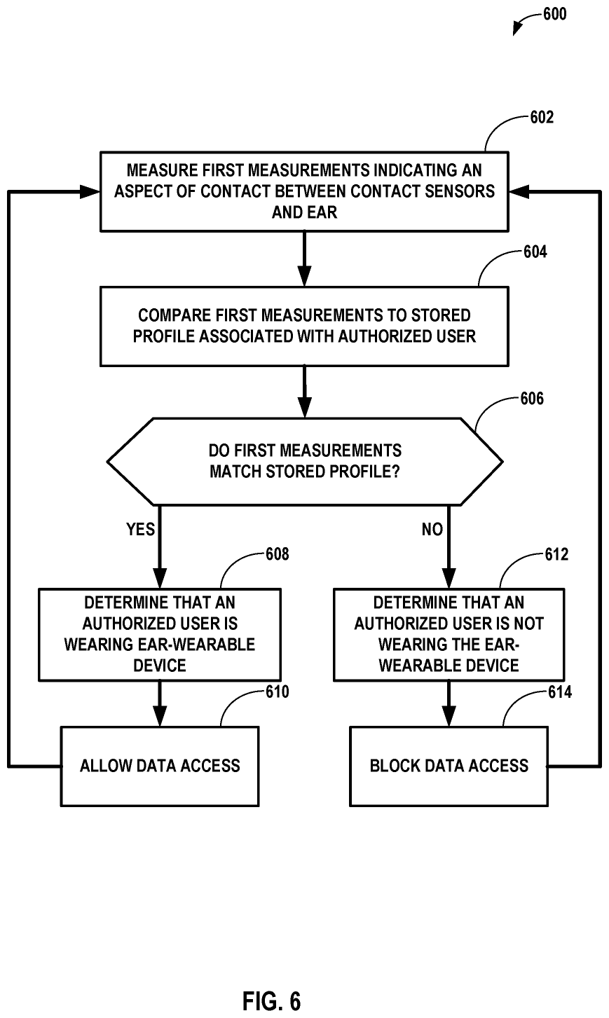

FIG. 6 is a flowchart illustrating an example operation 600 of this disclosure. Other examples of this disclosure may include more, fewer, or different actions. A plurality of contact sensors 236 coupled to an ear-wearable device are configured to measure first measurements indicative of an aspect of contact between the respective contact sensor and an ear of a user (602). For example, contact sensors 236 may include a plurality of electrodes, piezoelectric sensors, or other contact sensors configured to measure a relative amount of contact (e.g., force, pressure, surface area) between each respective contact sensor and the skin of the ear canal 446 of user 104.

Processor(s) 208 of hearing instrument 200 are configured to determine whether an authorized user is wearing hearing instrument 200, based at least in part on the first measurements generated by the contact sensors. For example, processor(s) 208 receive (e.g., retrieve from memory 202) a stored profile including a set of previous contact-sensor measurements for an authorized user. Processor(s) 208 may compare the first measurements to the stored profile (604), to determine whether the first measurements match (e.g., are within a threshold tolerance from) the previous contact-sensor measurements of the stored profile (606). If the first measurements match the stored profile (the “YES” branch from 606), processor(s) 208 determine that an authorized user is wearing hearing instrument 200 (e.g., current user 104 is identified as the authorized user from the stored profile (608). Responsive to determining that user 104 is an authorized user, processor(s) 208 may perform one or more actions to allow the authorized user access to data via one or more devices (610). For example, processor(s) 208 may enable hearing instrument 200 to receive and/or output audio data. In other examples, processor(s) 208 may output a signal to unlock a computing device for the authorized user. In other examples, processor(s) 208 may output a signal authorizing a data transfer between two or more computing devices (610). For example, processor(s) 208 may output a signal authorizing a digital payment from a personal computing device to a second computing device or system in response to identifying user 104.

Conversely, if the first measurements do not match the stored profile (the “NO” branch from 606), processor(s) 208 determine that an authorized user is not wearing hearing instrument 200 (e.g., processor(s) 208 fail to identify current user 104 as an authorized user of hearing instrument 200) (612). Responsive to determining that an authorized user is not wearing hearing instrument 200, processor(s) 208 may perform one or more actions to block or prevent current user 104 from accessing data via one or more devices (614). For example, processor(s) 208 may disable hearing instrument 200 such that it cannot receive audio data from another device and/or cannot output audio via a speaker of receiver 206. In some examples, processor(s) 208 may output a signal to lock an external computing device (e.g., a smartphone, laptop, tablet, etc.), such that additional authentication (e.g., a password or fingerprint) is required to access functionality on the device. In some examples, processor(s) 208 may output a signal causing an external computing device (e.g., laptop, smartphone, etc.) to erase data and/or disable any or all communication other devices, such as a remote computing device or system. In some examples, processor(s) 208 may output a signal preventing hearing instrument 200 from communicating (e.g., transferring data with) a second hearing instrument. In some examples, processor(s) 208 may output a signal preventing additional sensors 238 from measuring or transferring other biometric data. In some examples, processor(s) 208 may output a signal to disable a positioning system (e.g., GPS) for one or more electronic devices.

In some examples, system 100 may perform the actions of operation 600 at a pre-determined frequency. For example, contact sensors 236 may automatically generate biometric measurements (602) every few seconds or minutes, or essentially continuously, in order to ensure that an authorized user is wearing ear-wearable device 200. In some examples, system 100 may be configured such that an external computing device (e.g., smartphone, laptop, etc.) may require periodic ping messages from hearing instrument 200 in order to remain authenticated (e.g., unlocked, activated). For example, if a connected smartphone does not receive a “positive” identification update from hearing instrument 200 every few seconds, or alternatively, receives a “negative” identification update from hearing instrument 200, the smartphone may be configured to automatically lock or otherwise disable data access or other functionality.

The following numbered clauses provide some examples of this disclosure.

Clause 1: In some examples, an ear-wearable device includes: a shell shaped for wearing in an ear of a user; a plurality of contact sensors coupled to the shell, each respective contact sensor of the plurality of contact sensors configured to generate first measurements indicative of an aspect of contact between the respective contact sensor and the ear of the user; and processing circuitry configured to determine whether an authorized user is wearing the ear-wearable device based at least in part on the first measurements generated by the contact sensors.

Clause 2: In some examples of the ear-wearable device of clause 1, the processing circuitry is configured to determine whether an authorized user is wearing the ear-wearable device by comparing the first measurements to a stored profile associated with the authorized user.

Clause 3: In some examples of the ear-wearable device of clause 1 or clause 2, the plurality of contact sensors includes at least one electrode.

Clause 4: In some examples of the ear-wearable device of any of clauses 1-3, the plurality of contact sensors includes at least one piezoelectric sensor.

Clause 5: In some examples of the ear-wearable device of any of clauses 1-4, the device further includes at least one photoplethysmography (PPG) sensor configured to generate second measurements indicative of a blood perfusion surrounding the ear, wherein the processing circuitry is configured to determine whether an authorized user is wearing the ear-wearable device based at least in part on the first measurements and the second measurements.

Clause 6: In some examples of the ear-wearable device of any of clauses 1-4, the device further includes at least one photoplethysmography (PPG) sensor configured to generate second measurements indicative of a blood perfusion surrounding the ear, wherein the processing circuitry is configured to determine whether the ear-wearable device is correctly placed within the ear based at least in part on the second measurements.

Clause 7: In some examples of the ear-wearable device of any of clauses 1-6, the device further includes at least two temperature sensors configured to generate third measurements indicative of a temperature gradient surrounding the ear, wherein the processing circuitry is configured to determine whether the ear-wearable device is correctly placed within the ear of the user based at least in part on the third measurements.

Clause 8: In some examples of the ear-wearable device of any of clauses 1-7, the processing circuitry is further configured to: determine that the authorized user is wearing the ear-wearable device; and allow the authorized user access to data in response to determining that the authorized user is wearing the ear-wearable device.

Clause 9: In some examples of the ear-wearable device of clause 8, the processing circuitry is further configured to allow the authorized user access to data by enabling a data transfer between a first device and a second device.

Clause 10: In some examples of the ear-wearable device of clause 9, the first device is the ear-wearable device, and the second device is a computing device.

Clause 11: In some examples of the ear-wearable device of clause 9, the first device is a first computing device, and the second device is a second computing device.

Clause 12: In some examples of the ear-wearable device of clause 8, the processing circuitry is configured to allow the authorized user access to data by unlocking a smartphone.

Clause 13: In some examples of the ear-wearable device of any of clauses 1-12, the ear-wearable device includes a hearing aid or an earbud.

Clause 14: In some examples of the ear-wearable device of any of clauses 1-13, the ear-wearable device is configured to output an emitted sound; the ear-wearable device further includes a microphone configured to generate fourth measurements indicative of a reflected sound; and the processing circuitry is configured to determine whether the authorized user is wearing the ear-wearable device based at least in part on the fourth measurements.

Clause 15: In some examples of the ear-wearable device of clause 14, the fourth measurements indicate at least one audio characteristic including a detection delay from the emitted sound; a frequency spread of the reflected sound; a resonance of the reflected sound; or an amplitude attenuation between the emitted sound and the reflected sound.

Clause 16: In some examples of the ear-wearable device of any of clauses 1-15, the plurality of contact sensors are further configured to generate fifth measurements indicative of a change in an aspect of contact between the between the respective contact sensor and the ear of the user during movement of a jaw of the user; and the processing circuitry is configured to determine whether the authorized user is wearing the ear-wearable device based at least in part on the first measurements and the fifth measurements.

Clause 17: In some examples of the ear-wearable device of any of clauses 1-16, at least one sensor of the plurality of contact sensors is disposed on a seal area of the shell.

Clause 18: In some examples, a method of determining whether an authorized user is wearing an ear-wearable device includes: measuring, by a plurality of contact sensors disposed on a shell of the ear-wearable device, first measurements indicative of an aspect of contact between the respective contact sensor and an ear of a user; and determining, by processing circuitry, whether the authorized user is wearing the ear-wearable device based at least in part on the first measurements.

Clause 19: In some examples of the method of clause 18, determining whether the authorized user is wearing the ear-wearable device includes comparing the first measurements to a stored profile associated with the authorized user.

Clause 20: In some examples of the method of clause 18 or clause 19, the plurality of contact sensors includes at least one electrode.

Clause 21: In some examples of the method of any of clauses 18-20, the plurality of contact sensors includes at least one piezoelectric sensor.

Clause 22: In some examples of the method of any of clauses 18-21, the method further includes generating, by at least one photoplethysmography (PPG) sensor, second measurements indicative of a blood perfusion surrounding the ear; wherein determining whether an authorized user is wearing the ear-wearable device includes determining whether an authorized user is wearing the ear-wearable device based at least in part on the first measurements and the second measurements.

Clause 23: In some examples of the method of any of clauses 18-22, the method further includes: generating, by at least one photoplethysmography (PPG) sensor, second measurements indicative of a blood perfusion surrounding the ear; and determining, by the processing circuitry, whether the ear-wearable device is correctly placed within the ear based at least in part on the second measurements.

Clause 24: In some examples of the method of any of clauses 18-23, the method further includes generating, by at least two temperature sensors, third measurements indicative of a temperature gradient surrounding the ear; wherein determining whether the ear-wearable device is correctly placed within the ear of the user includes determining whether the ear-wearable device is correctly placed within the ear of the user based at least in part on the third measurements.

Clause 25: In some examples of the method of any of clauses 18-24, the method further includes: determining that the authorized user is wearing the ear-wearable device; and allowing the authorized user access to data in response to determining that the authorized user is wearing the ear-wearable device.

Clause 26: In some examples of the method of clause 25, allowing the authorized user access to data includes enabling a data transfer between a first device and a second device.

Clause 27: In some examples of the method of clause 26, the first device is the ear-wearable device, and the second device is a computing device.

Clause 28: In some examples of the method of clause 26, the first device is a first computing device, and the second device is a second computing device.

Clause 29: In some examples of the method of clause 25, allowing the authorized user access to data includes unlocking a smartphone.

Clause 30: In some examples of the method of any of clauses 18-29, the ear-wearable device includes a hearing aid or an earbud.

Clause 31: In some examples of the method of any of clauses 18-30, the method further includes: outputting, by the ear-wearable device, an emitted sound; and generating, by a microphone, fourth measurements indicative of a reflected sound; wherein determining whether the authorized user is wearing the ear-wearable device includes determining whether the authorized user is wearing the ear-wearable device based at least in part on the fourth measurements.

Clause 32: In some examples of the method of clause 31, the fourth measurements indicate at least one audio characteristic including: a detection delay from the emitted sound; a frequency spread of the reflected sound; a resonance of the reflected sound; or an amplitude attenuation between the emitted sound and the reflected sound.

Clause 33: In some examples of the method of any of clauses 18-32, the method further includes generating, by the plurality of contact sensors, fifth measurements indicative of a change in an aspect of contact between the between the respective contact sensor and the ear of the user during movement of a jaw of the user; wherein determining whether the authorized user is wearing the ear-wearable device includes determining whether the authorized user is wearing the ear-wearable device based at least in part on the first measurements and the fifth measurements.

Clause 34: In some examples of the method of any of clauses 18-33, at least one sensor of the plurality of contact sensors is disposed on a seal area of the shell.

In this disclosure, ordinal terms such as “first,” “second,” “third,” and so on, are not necessarily indicators of positions within an order, but rather may be used to distinguish different instances of the same thing. Examples provided in this disclosure may be used together, separately, or in various combinations. Furthermore, with respect to examples that involve personal data regarding a user, it may be required that such personal data only be used with the permission of the user.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

1. An ear-wearable device comprising:
a shell shaped for wearing in an ear of a user;
a plurality of contact sensors coupled to the shell, each respective contact sensor of the plurality of contact sensors configured to generate first measurements indicative of an aspect of contact between the respective contact sensor and the ear of the user; and
processing circuitry configured to determine whether an authorized user is wearing the ear-wearable device based at least in part on the first measurements generated by the contact sensors.

2. The ear-wearable device of claim 1, wherein the processing circuitry is configured to determine whether an authorized user is wearing the ear-wearable device by comparing the first measurements to a stored profile associated with the authorized user.

3. The ear-wearable device of claim 1, wherein the plurality of contact sensors comprises at least one electrode or at least one piezoelectric sensor.

4. (canceled)

5. The ear-wearable device of claim 1, further comprising at least one photoplethysmography (PPG) sensor configured to generate second measurements indicative of a blood perfusion surrounding the ear, wherein the processing circuitry is configured to determine whether an authorized user is wearing the ear-wearable device based at least in part on the first measurements and the second measurements.

6. The ear-wearable device of claim 1, further comprising at least one photoplethysmography (PPG) sensor configured to generate second measurements indicative of a blood perfusion surrounding the ear, wherein the processing circuitry is configured to determine whether the ear-wearable device is correctly placed within the ear based at least in part on the second measurements.

7. The ear-wearable device of claim 1, further comprising at least two temperature sensors configured to generate third measurements indicative of a temperature gradient surrounding the ear, wherein the processing circuitry is configured to determine whether the ear-wearable device is correctly placed within the ear of the user based at least in part on the third measurements.

8. The ear-wearable device of claim 1, wherein the processing circuitry is further configured to:
determine that the authorized user is wearing the ear-wearable device; and
allow the authorized user access to data in response to determining that the authorized user is wearing the ear-wearable device.

9. The ear-wearable device of claim 8, wherein the processing circuitry is further configured to allow the authorized user access to data by enabling a data transfer between a first device and a second device, wherein:
the first device is the ear-wearable device, and the second device is a computing device, or
the first device is a first computing device, and the second device is a second computing device.

10-11. (canceled)

12. The ear-wearable device of claim 8, wherein the processing circuitry is configured to allow the authorized user access to data by unlocking a smartphone or other device.

13. (canceled)

14. The ear-wearable device of claim 1, wherein:
the ear-wearable device is configured to output an emitted sound;
the ear-wearable device further comprises a microphone configured to generate fourth measurements indicative of a reflected sound; and
the processing circuitry is configured to determine whether the authorized user is wearing the ear-wearable device based at least in part on the fourth measurements.

15. The ear-wearable device of claim 14, wherein the fourth measurements indicate at least one audio characteristic comprising:
a detection delay from the emitted sound;
a frequency spread of the reflected sound;
a resonance of the reflected sound; or
an amplitude attenuation between the emitted sound and the reflected sound.

16. The ear-wearable device of claim 1, wherein:
the plurality of contact sensors are further configured to generate fifth measurements indicative of a change in an aspect of contact between the between the respective contact sensor and the ear of the user during movement of a jaw of the user; and
the processing circuitry is configured to determine whether the authorized user is wearing the ear-wearable device based at least in part on the first measurements and the fifth measurements.

17. The ear-wearable device of claim 1, wherein at least one sensor of the plurality of contact sensors is disposed on a seal area of the shell.

18. A method of determining whether an authorized user is wearing an ear-wearable device, the method comprising:
measuring, by a plurality of contact sensors disposed on a shell of the ear-wearable device, first measurements indicative of an aspect of contact between the respective contact sensor and an ear of a user; and
determining, by processing circuitry, whether the authorized user is wearing the ear-wearable device based at least in part on the first measurements.

19. The method of claim 18, wherein determining whether the authorized user is wearing the ear-wearable device comprises comparing the first measurements to a stored profile associated with the authorized user.

20-21. (canceled)

22. The method of claim 18, further comprising:
generating, by at least one photoplethysmography (PPG) sensor, second measurements indicative of a blood perfusion surrounding the ear; wherein determining whether an authorized user is wearing the ear-wearable device comprises determining whether an authorized user is wearing the ear-wearable device based at least in part on the first measurements and the second measurements.

23. The method of claim 18, comprising:
generating, by at least one photoplethysmography (PPG) sensor, second measurements indicative of a blood perfusion surrounding the ear; and
determining, by the processing circuitry, whether the ear-wearable device is correctly placed within the ear based at least in part on the second measurements.

24. The method of claim 18, further comprising:
generating, by at least two temperature sensors, third measurements indicative of a temperature gradient surrounding the ear; wherein determining whether the ear-wearable device is correctly placed within the ear of the user comprises determining whether the ear-wearable device is correctly placed within the ear of the user based at least in part on the third measurements.

25. The method of claim 18, further comprising:
determining that the authorized user is wearing the ear-wearable device; and
allowing the authorized user access to data in response to determining that the authorized user is wearing the ear-wearable device.

26. The method of claim 25, wherein allowing the authorized user access to data comprises enabling a data transfer between a first device and a second device,. wherein:
the first device is the ear-wearable device, and the second device is a computing device, or
the first device is a first computing device, and the second device is a second computing device.

27-30. (canceled)

31. The method of claim 18, further comprising:
outputting, by the ear-wearable device, an emitted sound; and
generating, by a microphone, fourth measurements indicative of a reflected sound; wherein
determining whether the authorized user is wearing the ear-wearable device comprises determining whether the authorized user is wearing the ear-wearable device based at least in part on the fourth measurements.

32. The method of claim 31, wherein the fourth measurements indicate at least one audio characteristic comprising:
a detection delay from the emitted sound;
a frequency spread of the reflected sound;
a resonance of the reflected sound; or
an amplitude attenuation between the emitted sound and the reflected sound.

33. The method of claim 18, further comprising:
generating, by the plurality of contact sensors, fifth measurements indicative of a change in an aspect of contact between the between the respective contact sensor and the ear of the user during movement of a jaw of the user; wherein
determining whether the authorized user is wearing the ear-wearable device comprises determining whether the authorized user is wearing the ear-wearable device based at least in part on the first measurements and the fifth measurements.

34. (canceled)
